(19) United States
(12) Patent Application Publication
(10) Pub. No.: US 2024/0358427 A1
(43) Pub. Date: Oct. 31, 2024

(54) STEERABLE RADIOFREQUENCY WIRE

(71) Applicant: Boston Scientific Medical Device Limited, Galway (IE)

(72) Inventors: Lauren Koon, Mississauga (CA); Gareth Davies, Toronto (CA)

(21) Appl. No.: 18/644,525

(22) Filed: Apr. 24, 2024

Related U.S. Application Data
(60) Provisional application No. 63/499,113, filed on Apr. 28, 2023.

(51) Int. Cl. A61B 18/12; A61B 18/00; A61B 18/14

(52) U.S. Cl. CPC A61B 18/1206; A61B 18/14; A61B 2018/00577; A61B 2018/126; A61B 2018/1425

(57) ABSTRACT

A medical device for treating a tumor includes an elongate body having a proximal portion, a distal portion, and an outer diameter. The medical device includes a steering mechanism configured to deflect the distal portion of the elongate body. A plurality of ablation electrodes are located on the distal portion. The ablation electrodes are adapted to deliver RF energy to the tumor. A tip electrode is located on the distal portion and the tip electrode is adapted to deliver sufficient RF energy to puncture the tumor. The distal portion is sufficiently stiff to enable the tip electrode to puncture the tumor.

### CROSS REFERENCE TO RELATED APPLICATIONS

The present application claims priority of U.S. Provisional Patent Application 63/499,113 entitled “STEERABLE RADIOFREQUENCY WIRE,” filed Apr. 28, 2023, which is hereby incorporated by reference in its entirety.

### TECHNICAL FIELD

The present invention relates generally to methods and devices usable within the body of a patient. More specifically, the present invention is concerned with a steerable medical device that can deliver radiofrequency (RF) energy to tissue.

### BACKGROUND

Treatment of deep-seated tumors is often treated by radiofrequency ablation (RFA). For this therapy, an ablation probe must be precisely placed, guided by precise and accurate needle placement. Accurate RFA probe placement results in optimal target coverage and minimal damage to surrounding healthy tissue.

However, undesired deflections of needles in the tissue increase with target depth and make needle placement more challenging. Typical causes of deflection are respiratory motions and unbalanced forces acting on the needle when traversing heterogenous structures. Additionally, when probing the tissue, which is a free anatomical structure, the application of force will cause the whole tissue structure to move, and the force will not be transmitted to the tissue.

### SUMMARY

Example 1 is a medical device for treating a tumor including an elongate body having a proximal portion, a distal portion, and an outer diameter. The medical device includes a steering mechanism configured to deflect the distal portion of the elongate body. A plurality of ablation electrodes are located on the distal portion and the ablation electrodes are adapted to deliver RF energy to the tumor. A tip electrode is located on the distal portion. The tip electrode is adapted to deliver sufficient RF energy to puncture the tumor and the distal portion is sufficiently stiff to enable the tip electrode to puncture the tumor.

Example 2 is the medical device of Example 1 wherein the elongate body includes a lumen extending from the proximal portion to the distal portion.

Example 3 is the medical device of any of Examples 1 or 2 wherein the tip electrode includes an outer surface having a diameter larger than the outer diameter of the elongate body.

Example 4 is the medical device of any of Examples 1 to 3 wherein the plurality of ablation electrodes are positioned proximal of the tip electrode.

Example 5 is the medical device of any of Examples 3 or 4 wherein the plurality of ablation electrodes are individually actuatable to change the size of a tumor ablation boundary.

Example 6 is the medical device of any of Examples 1 to 5 wherein the steering mechanism includes a rotatable knob.

Example 7 is the medical device of any of Examples 1 to 5 wherein the steering mechanism includes a pull wire or a push rod.

Example 8 is the medical device of Example 7 wherein the pull wire or push rod is attached to the elongate body offset from a longitudinal axis of the elongate body.

Example 9 is the medical device of Example 8 wherein the pull wire or push rod is attached to one of the plurality of ablation electrodes.

Example 10 is the medical device of any of Examples 1 to 9, further including a handle coupled to the proximal portion of the elongate body and operably coupled to the steering mechanism.

Example 11 is the medical device of Example 10 wherein the elongate body is removably coupled to the handle.

Example 12 is the medical device of any of Examples 1 to 11 further including a connector configured to removably couple the tip electrode to an RF generator.

Example 13 is the medical device of any of Examples 1 to 12 wherein the elongate body has a variable stiffness between the proximal portion and the distal portion.

Example 14 is the medical device of any of Examples 1 to 13 wherein the elongate body includes one or more cuts.

Example 15 is the medical device of any of Examples 1 to 14 wherein the proximal portion is formed of a first material and the distal portion is formed of a second material.

Example 16 is a radiofrequency medical device including an elongate body having a proximal portion, a distal portion, and an outer diameter. The medical device includes a tip electrode located on the distal portion, wherein the tip electrode includes an outer surface having a diameter larger than the outer diameter of the elongate body. A plurality of ablation electrodes are disposed along the distal portion, the ablation electrodes are configured to deliver ablation energy. The medical device includes a handle coupled to the proximal portion of the elongate body. A steering mechanism is operably associated with the handle. The steering mechanism is configured to deflect the distal portion of the elongate body.

Example 17 is the medical device of Example 16 wherein the elongate body includes a lumen extending from the proximal portion to the distal portion.

Example 18 is the medical device of Example 16 wherein the plurality of ablation electrodes are configured to deliver bipolar RF energy.

Example 19 is the medical device of Example 18 wherein the plurality of ablation electrodes are positioned proximal of the tip electrode.

Example 20 is the medical device of Example 19 wherein the plurality of ablation electrodes are individually actuatable to change the size of a tumor ablation boundary.

Example 21 is the medical device of Example 16 wherein the steering mechanism includes a rotatable knob.

Example 22 is the medical device of Example 16 wherein the steering mechanism includes a pull wire or a push rod.

Example 23 is the medical device of Example 22 wherein the pull wire or push rod is attached to the elongate body offset from a longitudinal axis of the elongate body.

Example 24 is the medical device of Example 23 wherein the pull wire or push rod is attached to one of the plurality of ablation electrodes.

Example 25 is the medical device of Example 16 wherein the handle is removably attached to the elongate body.

Example 26 is the medical device of Example 16, further including a connector configured to removably engage an RF generator.

Example 27 is the medical device of Example 16 wherein the elongate body has a variable stiffness between the proximal portion and the distal portion.

Example 28 is a system for performing a medical procedure. The system includes an elongate body having a proximal portion, a distal portion, and an outer diameter. A tip electrode is located on the distal portion, wherein the tip electrode includes an outer surface having a diameter larger than the outer diameter of the elongate body. The system includes a handle and a steering mechanism associated with the handle. The steering mechanism is configured to deflect the distal portion of the elongate body. The system includes a connector configured for electrically connecting with a radiofrequency generator.

Example 29 is the system of Example 28, further including a radiofrequency generator.

Example 30 is a method for treating a tumor in a patient. The method includes providing a treatment device including an elongate shaft having a distal tip electrode and a plurality of ablation electrodes. The treatment device also includes a steering mechanism for deflecting the distal tip. The method includes advancing the elongate shaft through a vessel of the patient to dispose the distal tip electrode proximal to the tumor. The distal portion of the elongate body is bent such that the distal tip electrode of the elongate body contacts a wall of the vessel. The method includes supplying RF energy to the distal tip electrode sufficient to puncture the wall of the vessel and urging the tip electrode forward to an internal portion of the tumor. Ablation energy is then supplied to the plurality of ablation electrodes.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a steerable RF wire in accordance with an embodiment of the present disclosure.

FIGS. 2A-2C are schematic illustrations of a medical procedure within tissue of a patient utilizing a steerable RF wire in accordance with an embodiment of the present disclosure.

FIG. 3 illustrates a steerable RF wire ablating tissue in accordance with the present disclosure.

FIGS. 4A and 4B illustrate a steerable RF wire in accordance with an embodiment of the present disclosure.

FIG. 5 illustrates a steerable RF wire in accordance with an embodiment of the present disclosure.

FIG. 6 illustrates an embodiment of a solid shaft for a steerable RF wire in accordance with an embodiment of the present disclosure.

FIG. 7 illustrates an embodiment of a hollow shaft for a steerable RF wire in accordance with an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

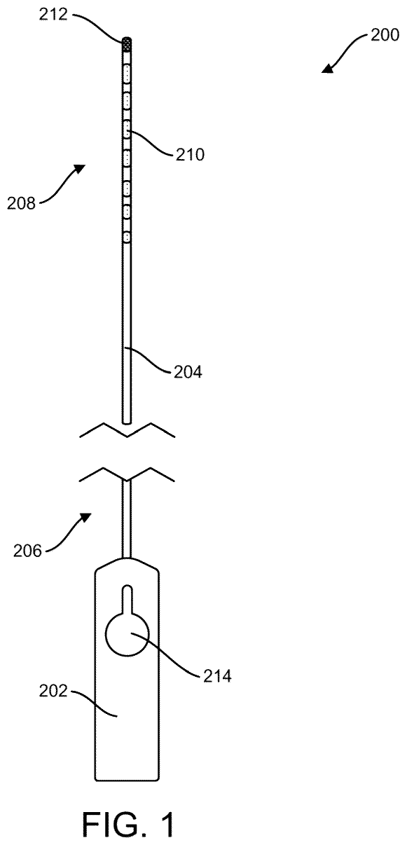

FIG. 1 illustrates a steerable RF wire 200 in accordance with an embodiment of the present disclosure. The steerable RF wire 200 is configured to be used as a piercing device as described above and/or for ablation of tissue, such as a tumor or other soft tissue. Soft tissue includes a tumor, tissue surrounding it, and tissue traversed while accessing the tumor. The steerable RF wire 200 can be used to create channels through tissue to arrive at a desired location within the tissue or to place the distal tip 212 at a desired location. In some aspects, the steerable RF wire 200 can be manipulated and steered through a patient's vasculature to a desired location.

The steerable RF wire 200 includes an elongate body 204 having a distal portion 208 and a proximal portion 206. The distal portion 208 includes a distal tip 212 which may be configured as a sharp needle tip, laser tip, or as a tip electrode. The elongate body 204 may be insulated or non-insulated. The proximal portion 206 is removably connected to a handle 202. The handle 202 includes a steering mechanism 214 such as a rotatable knob, linear actuator, or slider. The steering mechanism 214 allows for the distal portion 208 to articulate in one or more planes. In one aspect the distal portion 208 may articulate up to 11 degrees relative to an axis of the proximal portion 206. The elongate body 204 is adequately stiff to allow for advancement through body tissue and provides sufficient torque transfer from the handle 202 to the distal portion 208.

In one aspect, the handle 202 is detachable from the elongate body 204. The handle 202 can be detachable using a screw mechanism or two collets. Alternatively, the elongate body 204 may be configured to snap off or break from the handle once the steerable RF wire 200 reaches a desired location. The handle includes a connection configured to connect to RF generator.

The distal portion 208 includes a plurality of ablation electrodes 210. The plurality of ablation electrodes 210 are configured for ablating tissue or for use in electroanatomical mapping procedures. The plurality of ablation electrodes 210 can be formed as ring electrodes that are substantially flush with the outer surface of the elongate body 204. When formed as ring electrodes, the electrodes can be swaged on to the elongate body, after which, carbothane or tecoflex plastic, or similar may coat the elongate body.

In some aspects, each of the plurality of electrodes 210 are separately selectable or actuatable. This allows for a user to select which electrodes are used in a mapping procedure, or which electrodes are used in an ablation procedure to adjust the size of a tumor ablation boundary.

In some aspects, a distal electrode located at the distal tip 212 is optimized for cutting. The surface area of the distal electrode is maintained to a minimum, for example by minimizing the axial length of the electrode, in order to drive current density. In some aspects, the distal electrode may be larger in diameter than a portion of the elongate member 204. In some aspects, the elongate member 204 may include insulation up to the distal electrode. In other aspects, a heat resistant dielectric, such as a ceramic, may abut the electrode.

In some aspects, the plurality of ablation electrodes 210 have a larger surface area than the distal electrode located at the distal tip 212. The plurality of ablation electrodes 210 may include lengths significantly longer than the distal electrode located at the distal tip 212. In some aspects, one or more of the plurality of the ablation electrodes 210 could be used as return electrodes for use in a bipolar cutting arrangement.

The steerable RF wire 200 can be visible under ultrasound guidance or other imaging modality. A series of markers or surface enhancements may be provided on the elongate body 204 to identify different portions of the steerable RF wire 200 during a procedure.

The steerable RF wire 200 includes features both used for tissue puncture and tumor ablation. This combination of features allows for shorter procedure time. In current workflows, a needle is inserted to the tumor location, a stylet is removed from the needle and then RF probe is placed within the needle to provide ablation to the tumor. Using the steerable RF wire 200, the exchange of the stylet for the RF probe is eliminated.

In one aspect, the steerable RF wire 200 can be introduced into a patient's vasculature and guided through the vasculature near the target tissue (for example, a tumor). The enhanced steerability of the steerable RF wire 200 allows for more precision in device placement within the tumor.

The steerable RF wire 200 can be steered directionally to a desired ablation area. Once positioned near the desire ablation are, RF can be applied through the distal electrode located at the distal tip 212 and the steerable RF wire 200 can be advanced towards the target area without applying substantial pressure. This allows the steerable RF wire 200 to more accurately arrive at a desired site within a patient. Using RF in combination with a tip electrode ensures that minimal pressure is needed to advance the steerable RF wire 200 through tissue and does not cause the tissue to be displaced while the steerable RF wire 200 advances.

In one aspect, a tip electrode and/or the plurality of ablation electrodes 210 may be variable in length. For example, the tip electrode used to puncture tissue to allow for advancement of the steerable RF wire 200, may increase in length after puncture in order to have greater surface area for ablation. In this configuration, the steerable RF wire 200 benefits from a small electrode for the purpose of puncturing because less power can be used for the required current density. And during ablation, an electrode with greater surface area is capable of creating a larger lesion. This variability in electrode length may be achieved with a mechanism within the device to expose a greater electrode surface area. Alternatively, to control the surface area of the ablative electrode, a dilator, insulating sheath or needle may be used to cover the device, leaving only the desired length protruded for ablation.

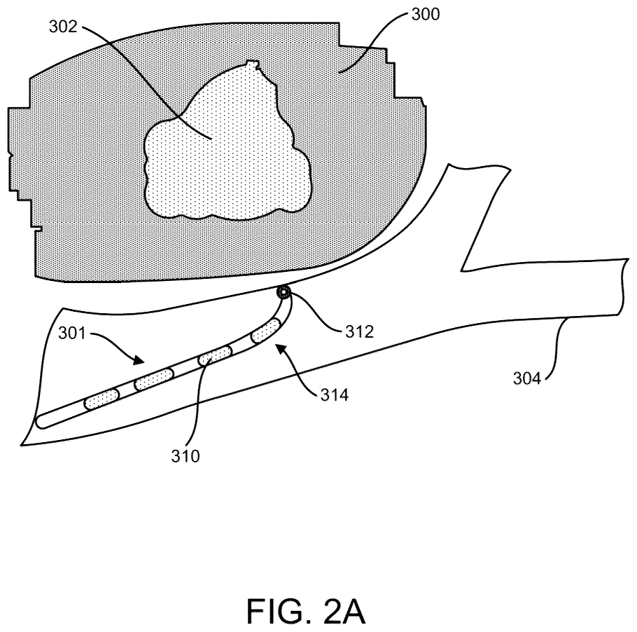

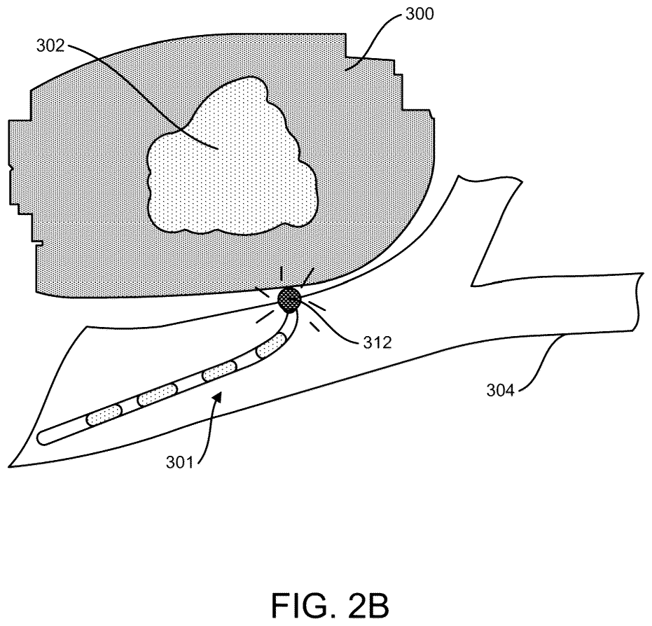

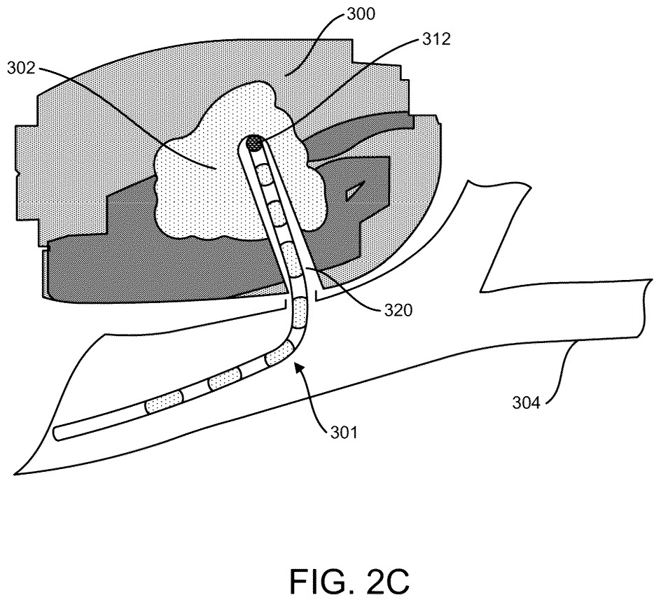

FIGS. 2A-2C are schematic illustrations of a medical procedure within tissue of a patient utilizing a steerable RF wire 301 in accordance with an embodiment of the present disclosure. FIG. 2A shows the steerable RF wire 301 located within a portion of a patient's vasculature 304. The steerable RF wire 301 can be guided through the vasculature 304 to a location adjacent a tissue 300 containing a tumor 302. For example, the tissue 300 may include a kidney, liver, heart, or other tissue. Once at a desired location, the distal tip 312 may be directed toward the tissue 300 by actuating a steering mechanism that changes the shape of a distal portion 314 of the steerable RF wire 301.

To direct the steerable RF wire 301 to the tissue 300, imaging such as ultrasound or fluoroscopy may be used. Portions of the steerable RF wire 301 may include markers or surface treatments to enhance visibility under an imaging modality. In some aspects, T-shaped marker bands may be included on the steerable RF wire 301 to help identify device directionality using fluoroscopy. In some aspects, the steerable RW wire 301 may include a radiopaque shaft, or a portion of the shaft may be radiopaque. In some aspects, the steerable RF wire 301 may include an echogenic coil such as a tungsten coil.

FIG. 2B illustrates applying RF energy to a tip electrode located at the distal tip 312 of the steerable RF wire 301 prior to advancement into the tissue 300. The RF energy is applied to the tip electrode by a conductor that passes through the body of the steerable RF wire 301. A proximal portion of the conductor may include a connector that connects to an RF generator.

FIG. 2C illustrates a channel 320 that is formed as the steerable RF wire 301 is advanced through the wall of the vasculature 304 and tissue 300 and into the tumor 302 while RF energy is applied to the tip electrode. Once at a desired location, confirmed by imaging, RF energy may no longer be applied to the tip electrode. Rather, a plurality of ablation electrodes 310 may be energized to ablate the tumor 302. As the steerable RF wire 301 exits after tumor ablation, the exit tract may be cauterized to prevent spreading of tumor cells.

The method of use will require RF to be delivered for puncture, but the waveform delivery will be changed for the ablative stage of the procedure, and then again for cautery upon withdrawal of the device from the tissue. The ablative energy used may be RF or pulsed field ablation. Another method that may be used may be bipolar ablation of the tumor where two electrodes are selected based on the tumor margins. The use of pulsed field ablation may be irreversible to create a similar effect as RF ablation. Alternatively, reversible RF ablation may prime tissue for uptake of chemical therapy.

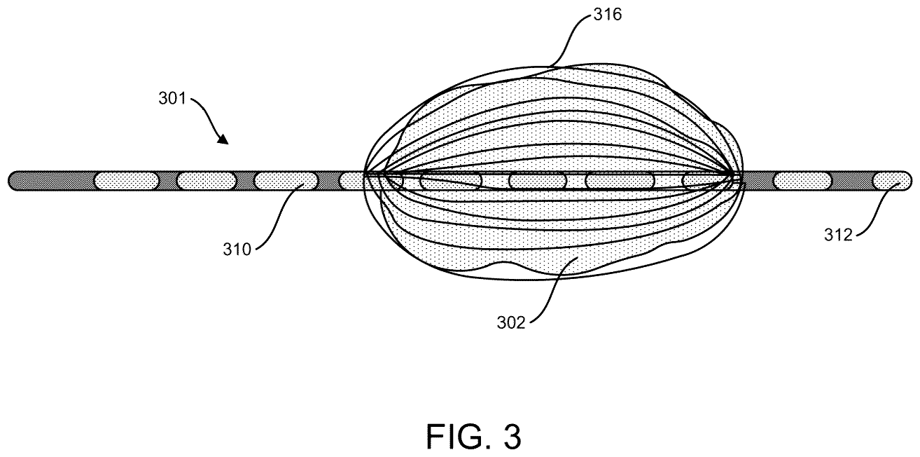

FIG. 3 illustrates the steerable RF wire 301 being used to ablate the tumor 302 in accordance with the present disclosure. As illustrated in FIG. 3, the plurality of ablation electrodes 310 are poisoned outside of and within the tumor 302. Bipolar RF energy is applied to selected ones of the plurality of ablation electrodes 310 in order to achieve a desired tumor ablation margin 316. By selecting which ablation electrodes 310 are used, healthy tissue can be prevented from being ablated or damaged.

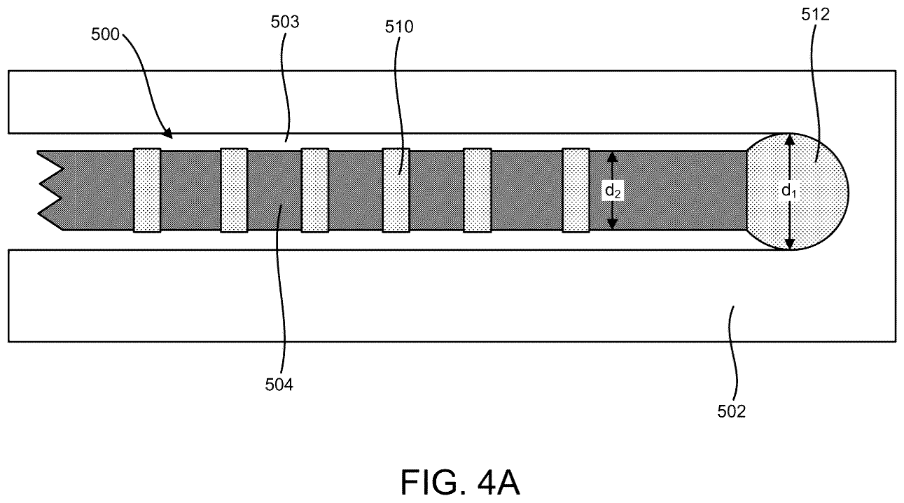

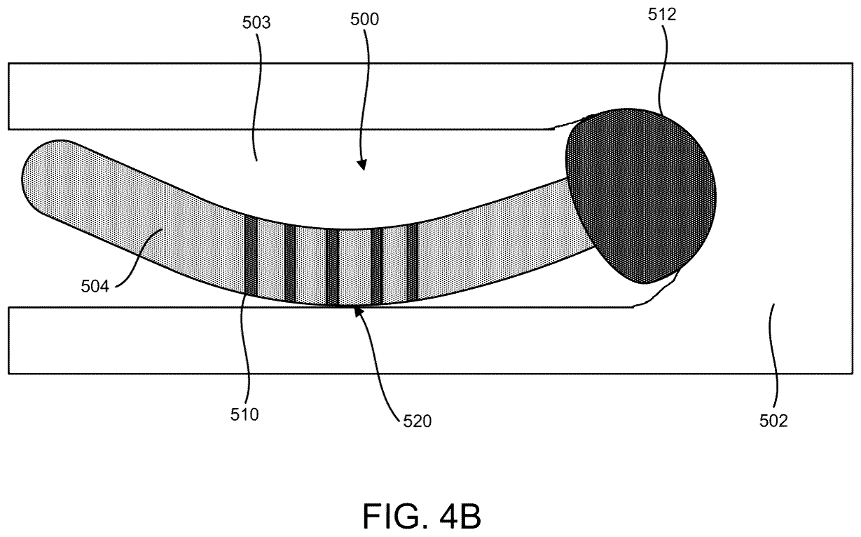

FIG. 4A illustrates a steerable RF wire 500 in accordance with an embodiment of the present disclosure. The steerable RF wire 500 includes an elongate body 504 having an outer surface. Positioned along the outer surface is a plurality of ablation electrodes 510. The distal tip includes a tip electrode 512. The tip electrode 512 is shown having a domed or spherical shape but may include other shapes. The diameter d1 of the tip electrode 512 is larger than a dimeter d2 of the elongate body 504.

The larger diameter of the tip electrode 512 dictates the channel 503 size through the solid tissue 502 when advanced therethrough. Because the elongate body 504 has a smaller diameter d2, the elongate body 504 can apply force to the tissue channel 503 wall and thus, the tip will be directionally steered.

FIG. 4B illustrates changing direction of the distal end of the steerable RF wire 500. In FIG. 4B an initial portion of the channel 503 though tissue 502 was formed by providing RF energy to the tip electrode 512 while advancing the steerable RF wire 500 in a linear direction. The larger diameter of the tip electrode 512 allows for the channel 503 to be slightly larger than the elongate body 504 of the steerable RF wire 500. In order to change direction of the distal end of the steerable RF wire 500, a distal portion is bent such that a portion of the elongate body contacts a wall of the initial portion of the channel 503 as illustrated at 520. This allows the distal end of the steerable RF wire 500 to be directed in a direction offset from the initial portion of the channel 503. As RF energy is applied to the tip electrode 512, the tip electrode 512 is urged forward by manipulating a proximal portion of the elongate body 504 to create a second portion of the channel 503 offset from the initial portion of the channel.

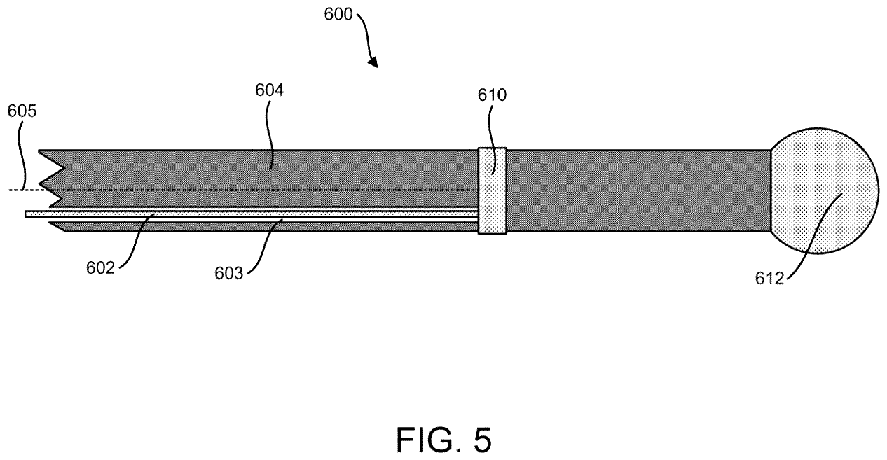

FIG. 5 illustrates a steerable RF wire 600 in accordance with an embodiment of the present disclosure. The steerable RF wire 600 includes an elongate body 604 having a distal tip electrode 612, and an ablation electrode 610. The steerable RF wire 600 includes a pull wire or push rod 602. The pull wire or push rod 602 extends from a proximal portion of the steerable RF wire 600 to the ablation electrode 610 and is attached to the ablation electrode 610. The attachment point of the pull wire or push rod 610 is offset from a longitudinal axis 605 of the steerable RF wire 600. In one aspect, the pull wire or push rod 610 may extend along an outer surface of the elongate body 604. In another aspect, the pull wire or push rod 610 may extend along a lumen 603 from the proximal portion to the ablation electrode 610.

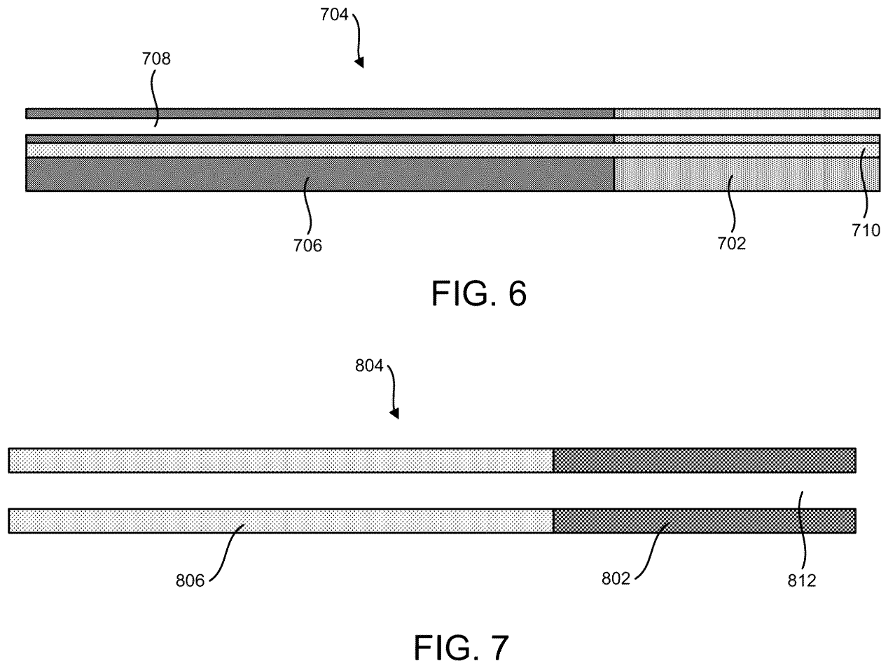

FIG. 6 illustrates an embodiment of a solid shaft 704 forming the elongate body for a steerable RF wire in accordance with an embodiment of the present disclosure. The shaft 704 includes a proximal portion 706 and a distal portion 702. A solid mandrel 710 extends along the length of the shaft and is surrounded by insulation such PTFE insulation or PI dip insulation.

The shaft 704 has variable stiffness along the length thereof to allow for deflection in the distal portion of the shaft and better force and torque transmission in a proximal portion of the shaft 704. In one aspect, the variable stiffness is provided by providing different insulations in the proximal portion 706 and the distal portion 702. In another aspect, the variable stiffness is provided by providing the mandrel with sections of variable stiffness. This may be done by modifying portions of the mandrel, such as by having portions having different diameters, or by forming the mandrel of different materials along a length thereof. The shaft 704 may include a lumen 708. The lumen provides a location for a pull wire or push rod, or conductors. In some aspects the lumen may include portions positioned 90 degrees to the deflection direction to prevent strain of wires.

FIG. 7 illustrates an embodiment of a hollow shaft 804 forming the elongate body for a steerable RF wire in accordance with an embodiment of the present disclosure. The hollow shaft 804 includes a lumen 812 extending form a proximal portion 806 to a distal portion 802. The lumen 812 allows for taking a biopsy of a tissue by creating a core with the lumen 812, and aspirating. The lumen 812 may be used as a conduit for a fluid agent, such as contrast, or for ablation (e.g. alcohol ablation). The lumen 812 may also contain one or more pull wire.

In some aspects, the elongate body of the steerable RF wire may include a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the elongate body. In some aspects, there may be a single cut that winds around an axis with a wider spacing between loops at the proximal portion and a larger spacing at the distal portion. The spacing and size of the cuts can be varied to achieve different flexibilities along the length of the elongate body.

In some aspects, a portion of the elongate body may be formed of a shape memory material, such as a shape memory polymer or a shape memory metal. This would allow the steerable RF wire to have a first shape at a first temperature, and a second shape at a second temperature. Shape transition may be initiated in the by inserting a heated solution or using electricity to heat a portion of the elongate body.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A radiofrequency medical device, comprising:
an elongate body having a proximal portion, a distal portion, and an outer diameter;
a tip electrode located on the distal portion, wherein the tip electrode includes an outer surface having a diameter larger than the outer diameter of the distal portion;
a plurality of ablation electrodes disposed along the distal portion, the ablation electrodes configured to delivery ablation energy;
a handle coupled to the proximal portion of the elongate body; and
a steering mechanism operably associated with the handle, the steering mechanism being configured to deflect the distal portion of the elongate body.

2. The medical device of claim 1, wherein the elongate body includes a lumen extending from the proximal portion to the distal portion.

3. The medical device of claim 1, wherein the plurality of ablation electrodes are configured to delivery bipolar RF energy.

4. The medical device of claim 3, wherein the plurality of ablation electrodes are positioned proximal of the tip electrode.

5. The medical device of claim 4, wherein the plurality of ablation electrodes are individually actuatable to change the size of a tumor ablation boundary.

6. The medical device of claim 1, wherein the steering mechanism includes a rotatable knob.

7. The medical device of claim 1, wherein the steering mechanism includes a pull wire or a push rod.

8. The medical device of claim 7, wherein the pull wire or push rod is attached to the elongate body offset from a longitudinal axis of the elongate body.

9. The medical device of claim 8, wherein the pull wire or push rod is attached to one of the plurality of ablation electrodes.

10. The medical device of claim 1, wherein the handle is removably attached to the elongate body.

11. The medical device of claim 1, further comprising:
a connector configured to removably engage an RF generator.

12. The medical device of claim 1, wherein the elongate body has a variable stiffness between the proximal portion and the distal portion.

13. A system for performing a medical procedure, comprising:
an elongate body having a proximal portion, a distal portion, and an outer diameter;
a tip electrode located on the distal portion, wherein the tip electrode includes an outer surface having a diameter larger than the outer diameter of the distal portion;
a handle;
a steering mechanism associated with the handle, the steering mechanism being configured to deflect the distal portion of the elongate body; and
a connector configured for electrically connecting with a radiofrequency generator.

14. The system of claim 13, further comprising:
a radiofrequency generator.

15. A method for treating a tumor in a patient, the method comprising:
providing a treatment device including an elongate shaft having a distal tip electrode and a plurality of ablation electrodes, the treatment device further including a steering mechanism for deflecting the distal tip;
advancing the elongate shaft through a vessel of the patient to dispose the distal tip electrode proximal to the tumor;
bending a distal portion of the elongate body such that the distal tip electrode of the elongate body contacts a wall of the vessel; and
supplying RF energy to the distal tip electrode sufficient to puncture the wall of the vessel;
urging the tip electrode forward to an internal portion of the tumor; and
supplying ablation energy to the plurality of ablation electrodes.
